(19) United States
(12) Patent Application Publication
(10) Pub. No.: US 2024/0041885 A1
(43) Pub. Date: Feb. 8, 2024

(54) CELLULAR HEALTH NUTRITIONAL PRODUCT

(71) Applicant: GLAXOSMITHKLINE CONSUMER HEALTHCARE HOLDINGS (US) LLC, Wilmington, DE (US)

(72) Inventors: Caroline Felicity BULL, Adelaide, , South Australia (AU); Michael FENECH, North Brighton, South Australia (AU); Scott William POXON, Richmond, VA (US); Bernardus Jan-Willem VAN KLINKEN, Warren, NJ (US)

(21) Appl. No.: 18/257,429

(22) PCT Filed: Dec. 20, 2021
(86) PCT No.: PCT/US2021/064330

§ 371 Date: Jun. 14, 2023

Related U.S. Application Data
(60) Provisional application No. 63/128,417, filed on Dec. 21, 2020.

(51) Int. Cl. A61K 31/519; A61K 31/714; A61K 31/593; A61K 33/30; A61K 31/26; A61K 31/352; A61P 39/00

(52) U.S. Cl. CPC A61K 31/519; A61K 31/714; A61K 31/593; A61K 33/30; A61K 31/26; A61K 31/352; A61P 39/00

(57) ABSTRACT

A nutritional supplement composition formulated to minimize DNA damage and improve DNA repair includes Folate, Vitamin B12, Vitamin D and Zinc.

### BACKGROUND

### 1. Field of the Disclosure

The present disclosure relates to a cellular health formulation. More particularly, the present disclosure relates to such a cellular DNA replication and repair formulation having nutrients for reducing cellular biomarkers of DNA damage and improving cellular regenerative capacity in individuals.

### 2. Brief Description of Related Art

As a person ages, the person's body shows signs of aging. Signs of aging include genomic instability, telomere attrition, epigenetic alterations, loss of proteostasis, deregulated nutrient sensing, mitochondrial dysfunction, cellular senescence, stem cell exhaustion and altered intercellular communication.

People are living longer. However, the burden of age-related diseases is increasing. Further, the cost of medical intervention, and the associated social and personal burden, is becoming difficult and perhaps unsustainable. Therefore, there is an increasing need to address the fundamental pathologies that cause age-related diseases.

In cells, increased DNA damage at the chromosomal, telomere, mitochondrial DNA, DNA base and DNA methylation levels are all associated with increased risk of developmental and degenerative disease and accelerated aging. Minimizing the excessive accumulation of DNA damage can by facilitated by optimal diet and lifestyle of an individual that is important to health and well-being.

Maintenance of genome integrity is dependent on good nutrition to provide the substrates and cofactors required to synthesize nucleotides, accurately replicate and repair DNA, and minimize or prevent endogenous and exogenous genotoxicants from harming the genome. Over the past two decades it has become evident that overt deficiency, as well as sub-clinical deficiencies in micronutrients involved in these processes, can cause as much genome damage as that induced by environmental and lifestyle genotoxins. Also, these deficiencies increase susceptibility to these hazards.

Studies have consistently indicated that a higher rate of chromosomal damage, such as micronuclei and chromosome breaks or rearrangements, as well as telomere shortening, predict an increased risk for age-related diseases, such as cancer and cardiovascular disease.

It has been demonstrated in several intervention studies that chromosomal DNA damage and telomere shortening can be reduced by appropriate dietary change and/or micronutrient supplementation. Further, healthy aging at the most fundamental level can be promoted by optimizing micronutrient intake.

### SUMMARY

The present disclosure provides a nutritional supplement composition that has a combination of micronutrients and phytonutrients determined to be essential for supporting cellular DNA replication and DNA repair functions.

The present disclosure also provides such a nutritional supplement composition that enables optimal cellular maintenance, for example chromosomal and telomere DNA integrity.

The present disclosure further provides such a nutritional supplement composition that has a combination of micronutrients selected to promote genome integrity and, preferably, also cellular health and regenerative capacity.

The present disclosure still further provides such a nutritional supplement composition that includes the following nutrients: Folate, Vitamin B12, Vitamin D and Zinc alone, or in combination with one or both of Sulforaphane, Grape Seed Proanthocyanidins, and Broccoli sprout or seed extract.

The present disclosure provides a method to increase DNA replication comprising administering such a nutritional supplement composition that includes the following nutrients: Folate, Vitamin B12, Vitamin D and Zinc alone, or in combination with one or both of Sulforaphane, Grape Seed Proanthocyanidins, and Broccoli sprout or seed extract.

The present disclosure provides a method to increase cellular efficiency of DNA repair comprising administering such a nutritional supplement composition that includes the following nutrients: Folate, Vitamin B12, Vitamin D and Zinc alone, or in combination with one or both of Sulforaphane, Grape Seed Proanthocyanidins, and Broccoli sprout or seed extract.

The present disclosure provides a method to induce a cell to increase cellular methylation of DNA comprising administering such a nutritional supplement composition that includes the following nutrients: Folate, Vitamin B12, Vitamin D and Zinc alone, or in combination with one or both of Sulforaphane, Grape Seed Proanthocyanidins, and Broccoli sprout or seed extract.

The present disclosure provides a method to increase telomere maintenance comprising administering such a nutritional supplement composition that includes the following nutrients: Folate, Vitamin B12, Vitamin D and Zinc alone, or in combination with one or both of Sulforaphane, Grape Seed Proanthocyanidins, and Broccoli sprout or seed extract.

The present disclosure provides a method to reduce cellular uridine DNA mutations comprising administering such a nutritional supplement composition that includes the following nutrients: Folate, Vitamin B12, Vitamin D and Zinc alone, or in combination with one or both of Sulforaphane, Grape Seed Proanthocyanidins, and Broccoli sprout or seed extract.

The present disclosure provides a method to reduce abnormal methylation of DNA in a cell, the method comprising administering such a nutritional supplement composition that includes the following nutrients: Folate, Vitamin B12, Vitamin D and Zinc alone, or in combination with one or both of Sulforaphane, Grape Seed Proanthocyanidins, and Broccoli sprout or seed extract.

The present disclosure provides a method to induce a cellular reduction in DNA strand breaks and base damage comprising administering such a nutritional supplement composition that includes the following nutrients: Folate, Vitamin B12, Vitamin D and Zinc alone, or in combination with one or both of Sulforaphane, Grape Seed Proanthocyanidins, and Broccoli sprout or seed extract.

The present disclosure provides a method for reducing inflammation by preventing micronucleus formation comprising administering such a nutritional supplement composition that includes the following nutrients: Folate, Vitamin B12, Vitamin D and Zinc alone, or in combination with one or both of Sulforaphane, Grape Seed Proanthocyanidins, and Broccoli sprout or seed extract.

In such a nutritional supplement composition, Folate can be present in an amount from 8 mcg to 1000 mcg, preferably in an amount from 50 mcg to 800 mcg, and more preferably in an amount from 120 mcg to 400 mcg.

In such a nutritional supplement composition, Vitamin B12 can be present in an amount from 0.04 mcg to 200 mcg, preferably in an amount from 1 mcg to 50 mcg, and more preferably in an amount from 6 mcg to 20 mcg.

In such a nutritional supplement composition, Vitamin D3 can be present in an amount from 12 IU to 4000 IU, preferably in an amount from 100 IU to 3000 IU, and more preferably in an amount from 600 IU to 2000 IU.

In such a nutritional supplement composition, Zinc can be present in an amount from 0.18 mg to 40 mg, preferably in an amount from 2 mg to 30 mg, and more preferably in an amount from 6 mg to 20 mg.

In such a nutritional supplement composition, Sulforaphane can be present in an amount from 0.5 mg to 250 mg, preferably in an amount from 3 mg to 100 mg, and more preferably in an amount from 7 mg to 25.5 mg.

In such a nutritional supplement composition, Grape Seed Proanthocyanidin can be present in an amount from 5.6 mg to 3000 mg, preferably in an amount from 10 mg to 1000 mg, and more preferably in an amount from 84 mg to 280 mg.

Among the countless known vitamins, minerals, peptides and phytonutrients, the combination of nutrients, namely Folate, Vitamin B12, Vitamin D and Zinc alone or in combination with one or both of Sulforaphane, Grape Seed Proanthocyanidins and Broccoli sprout or seed extract, has surprisingly been found effective to promote genome integrity and, preferably, also cellular health and regenerative capacity. The cellular health and regenerative capacity is based on the level of DNA damage in humans measured using certain biomarkers that increase with age and oxidative stress and that are modifiable by dietary intervention.

These biomarkers include: (1) chromosome aberrations (in lymphocytes), (2) micronuclei (in lymphocytes and buccal cells), (3) DNA strand breaks measured by “comet” assay (in lymphocytes/leukocytes), (4) DNA oxidation measured as oxidized DNA bases (in lymphocytes/leukocytes) and (5) telomere length or telomerase activity (in lymphocytes/leukocytes). These biomarkers have been found to be the best validated biomarkers of genome integrity.

Without wishing to be bound by a single theory, the nutritional supplement compositions of the present disclosure improve genome integrity by the mechanism of micronutrients essential for the cellular functions of DNA replication and DNA repair, that enable optimal maintenance of chromosomal and telomere DNA integrity.

### BRIEF DESCRIPTION OF THE DRAWINGS

FIG. 1 is a summary of ingredients and mechanisms of action according to the present disclosure.

FIG. 2 shows examples of cellular damage at the DNA level.

FIG. 3 is a summary of the effect of supplementation with a formulation according to the present disclosure.

FIG. 4 is another summary of the effect of supplementation with a formulation according to the present disclosure.

FIG. 5 is yet another summary of the effect of supplementation with a formulation according to the present disclosure.

### DETAILED DESCRIPTION OF THE DISCLOSURE

It has been found by the present disclosure that it is possible to tuneup or improve cellular function of DNA metabolism by increasing the supply of a certain combination of micronutrients and phytonutrients required for nucleotide synthesis, DNA replication, DNA damage-sensing and signaling and DNA repair.

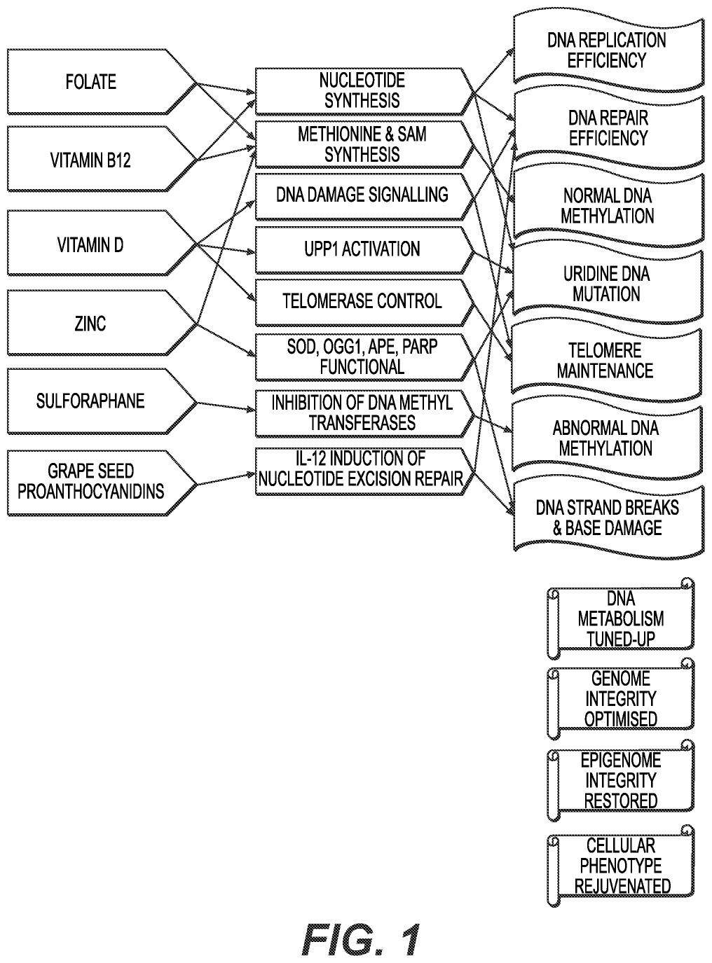

Referring to FIG. 1, the combination of micronutrients according to the present disclosure includes: Folate, Vitamin B12, Vitamin D, Zinc, Sulforaphane and Grape Seed Proanthocyanidins.

The combination supports nucleotide synthesis; methionine & SAM synthesis, DNA damage signaling; UPP1 activation; telomerase control; SOD, OGG1, APE, and PARP enzyme function.

The combination increases cellular DNA replication efficiency, normal DNA methylation, and telomere maintenance. The combination also decreases cellular uridine DNA mutation, abnormal DNA methylation, and DNA strand breaks and bas damage. Thus, the combination tunes cellular DNA metabolism, optimizes genome integrity, restores epigenome integrity, and rejuvenations cellular phenotypes.

The combination provides cellular support for cellular DNA replication and cellular DNA repair processes by increasing the supply of micronutrients required for nucleotide synthesis, DNA replication, DNA damage-sensing and signaling and DNA repair.

The combination play an important role in maintenance of DNA methylation patterns and their restoration through multiple mechanisms to optimize genome integrity, restore epigenome integrity and promote rejuvenation of the cellular phenotype.

As an example, one of the most important pathological base mutations is the incorporation of uridine into DNA due to excess dUTP. The inclusion in the combination of (i) Folate and Vitamin B12 is for the synthesis of 5,10-methylenetetrahydrofolate, the methyl donor that converts dUTP to dTTP and the inclusion in the combination of (ii) Vitamin D activates UPP1 required for the phosphorylytic cleavage of uridine and deoxyuridine to uracil and ribose- or deoxyribose-1-phosphate. These produced molecules are then utilized as carbon and energy sources or in the rescue of pyrimidine bases for nucleotide synthesis.

As another example, included in the combination is the stimulation by both grape seed anthocyanidins of IL-2 synthesis that activates nucleotide excision repair, with the latter also being supported by Zinc via its structural role in the enzymes OGG1 and APE which repair oxidized guanine and apurinic/apyrimidinic sites in DNA.

As yet another example, the combination of Folate, Vitamin B12 and Zinc in supports the synthesis of SAM required for appropriate maintenance of methylation of CpG sites in DNA to suppress expression of parasitic repeat sequences, such as LINE-1 DNA, and the involvement of Sulforaphane in reversing the inappropriate silencing of tumor-suppressor and other house-keeping genes caused by age-related epigenetic drift by its inhibition of DNA methyltransferases.

A nutritional supplement composition according to the present disclosure includes one or more vitamins, minerals, peptides, and/or phytonutrients.

Folate can be present in an amount from 8 mcg to 1000 mcg, preferably in an amount from 50 mcg to 800 mcg, and more preferably in an amount from 120 mcg to 400 mcg.

Vitamin B12 can be present in an amount from 0.04 mcg to 200 mcg, preferably in an amount from 1 mcg to 50 mcg, and more preferably in an amount from 6 mcg to 20 mcg.

Vitamin D3 can be present in an amount from 12 IU to 4000 IU, preferably in an amount from 100 IU to 3000 IU, and more preferably in an amount from 600 IU to 2000 IU.

Zinc can be present in an amount from 0.18 mg to 40 mg, preferably in an amount from 2 mg to 30 mg, and more preferably in an amount from 6 mg to 20 mg.

Sulforaphane can be present in an amount from 0.5 mg to 250 mg, preferably in an amount from 3 mg to 100 mg, and more preferably in an amount from 7 mg to 25.5 mg.

Grape Seed Proanthocyanidin can be present in an amount from 5.6 mg to 3000 mg, preferably in an amount from 10 mg to 1000 mg, and more preferably in an amount from 84 mg to 280 mg.

In an exemplary composition: Folate in the form of folic acid is in an amount from 8 mcg to 1000 mcg; Vitamin B12 is in an amount from 0.04 mcg to 200 mcg; Vitamin D3 is in an amount from 12 IU to 4000 IU; Zinc is in an amount from 0.18 mg to 40 mg; Sulforaphane is in an amount from 0.5 mg to 250 mg; and Grape Seed Proanthocyanidin is in an amount from 5.6 mg to 3000 mg.

In another exemplary composition: Folate in the form of folic acid is in an amount from 8 mcg to 1000 mcg; Vitamin B12 is in an amount from 0.04 mcg to 200 mcg; Vitamin D3 is in an amount from 12 IU to 4000 IU; and Zinc is in an amount from mg to 40 mg. Optionally, Sulforaphane is in an amount from 0.5 mg to 250 mg; and/or Grape Seed Proanthocyanidin is in an amount from 5.6 mg to 3000 mg are optional ingredients.

In still another exemplary composition: Folate in the form of folic acid is in an amount of about 120 mcg or about 400 mcg; Vitamin B12 is in an amount of about 6 mcg or about 20 mcg; Vitamin D3 is in an amount of about 600 IU or about 2000 IU; Zinc is in an amount of about 6 mg or about 20 mg; Sulforaphane is in an amount of about 7 mg or about 25.5 mg; and Grape Seed Proanthocyanidin is in an amount of about 84 mg or about 280 mg. In this composition, about means plus or minus 15%, preferably plus or minus 10%, more preferably plus or minus 7%, and most preferably plus or minus 5%.

In yet another exemplary composition: Folate in the form of folic acid is in an amount of about 120 mcg or about 400 mcg; Vitamin B12 is in an amount of about 6 mcg or about 20 mcg; Vitamin D3 is in an amount of about 600 IU or about 2000 iu; and Zinc is in an amount of about 6 mg or about 20 mg. Optionally, Sulforaphane is in an amount of about 7 mg or about 25.5 mg and/or Grape Seed Proanthocyanidin in an amount of about 84 mg or about 280 mg. In these compositions, about also means plus or minus 15%, preferably plus or minus 10%, more preferably plus or minus 7% and most preferably plus or minus 5%.

The above compositions have been surprisingly found to be effective at reducing DNA damage biomarkers and/or improving cellular regenerative capacity in individuals with clinical deficiency in the micronutrients. For example, these combinations of micronutrients in the compositions play an important role in maintenance of DNA methylation patterns and restoration. Moreover, it has been unexpectedly found that the combinations can ameliorate abnormal nucleotides and DNA replication stress resulting in efficient nucleotide synthesis. Further, these combinations can ameliorate one or more or all of: (1) stalling of DNA replication and increases in DNA breaks resulting in high fidelity DNA replication, (2) hypomethylated dysfunctional centromeres and abnormal gene expression resulting in proper maintenance of DNA methylation, (3) abnormally long or short dysfunctional telomeres resulting in normal telomere length maintenance, (4) aneuploidy that is an abnormal chromosome number resulting in accurate chromosomes segregation, (5) misrepair and/or lack of repair of DNA breaks or DNA adducts resulting in efficient and/or accurate DNA repair, and (6) silencing of DNA repair and cell cycle genes by DNA hypermethylation resulting in reversal of DNA hypermethylation.

The term “Folate” is intended to include all forms of folic acid, folinic acid and 5-Methyltetrahydrofolate.

Folate is important for DNA synthesis, stability and integrity and repair. Folate deficiency is known to cause chromosome aberrations and mitochondrial DNA deletions.

Folate in cells is present in various chemical forms. These chemical forms play essential roles in cellular metabolism by their ability to donate carbon atoms to synthesize important molecules required for cellular proliferation and DNA metabolism.

In the form 5-methyltetrahydrofolate, Folate donates its methyl group to homocysteine to synthesize methionine that is vital for protein synthesis and for the synthesis of SAM that is the common methyl donor required for several metabolic reactions. The most notable metabolic reaction is the maintenance of methylation at CpG islands in DNA, which controls gene expression and the structural integrity of the pericentromeric regions of chromosomes that is essential for accurate chromosome segregation during mitosis.

In the form 5,10-methylenetetrahydrofolate, Folate donates it carbon atom to deoxyuridine monophosphate (dUMP) to synthesize the nucleotide thymidine (dTTP) which is critical for DNA synthesis.

In the form 10-formyl tetrahydrofolate, Folate is essential for purine nucleotides (dATP, dGTP) that are required for DNA synthesis.

Many aspects of health are affected at the cellular and organ system level by Folate. When Folate is deficient cells cannot generate enough nucleotides to properly replicate DNA leading to DNA replication stress, DNA strand breaks and chromosome aberrations. Also when Folate is deficient the cells' capacity to maintain cytosine methylation is impaired and can result in altered gene expression and loss of chromatin structural integrity at the pericentric regions of chromosomes. Further, homocysteine is not converted to methionine resulting in hyper-homocystenemia. Consequently, genome integrity is diminished, the regenerative capacity of tissues is impaired, and toxic levels of homocysteine become more probable leading to accelerated aging of tissues, which can be manifested as anemia, neurodegenerative diseases, cardiovascular disease and cancer.

In present examples, Folate is in the form of folic acid.

Folic acid is the stable non-reduced, non-methylated, monoglutamated form of Folate commonly used in supplements which can be readily metabolized in the body to 5,10-methylenetetrahydrofolate; 5-methyltetrahydrofolate; and 10-formyl tetrahydrofolate.

Also, in examples, the dosage of Folate in the nutritional supplement composition is about 400 μg/day. At a dosage of about 400 μg/day, optimal genome stability and normal homocysteine concentration in the blood is achieved.

The term “Vitamin B12” includes all functionally equivalent forms thereof.

Vitamin B12 plays a role in nucleotide synthesis and DNA methylation. When Vitamin B12 is deficient, the enzymes MTR and methylmalonyl-CoA mutase (MCM) do not function efficiently. Consequently, the toxic metabolites homocysteine and methylmalonic acid increase in concentration, mitochondrial metabolism is impaired, and cells become unable to store and utilize Folate. The former is because cannot be converted to tetrahydrofolate, which is the form of Folate that can be polyglutamated and stored in cells, and is converted to 10-formyltetrahydrofolate and to 5,10-methylenetetrahydrofolate, which are required for nucleotide synthesis. Essentially Folate is not converted becomes it is “trapped” and unusable in the Folate-methionine one-carbon metabolism cycle when Vitamin B-12 is deficient.

Like a Folate deficiency, a Vitamin B-12 deficiency results in DNA replication stress, loss of genome integrity and hypomethylation of DNA resulting in accelerated aging and loss of regenerative capacity and megaloblastic anemia. Furthermore, Vitamin B12 deficiency is associated with increased risk of cardiovascular disease and neurodegenerative diseases, such as dementia, peripheral neuropathy and myelopathy caused by demyelination of neurons.

In present examples, Vitamin B12 is in the form of methyl-cobalamin and adenosyl-cobalamin. [or is it “The form used in the supplement will be cyanocobalamin which is readily converted to hydroxocobalamin, cyanocobalamin and adenosyl-cobalamin in vivo.”]

Methyl-cobalamin, in its reduced form, acts as cofactor for methionine synthase (MTR) to convert homocysteine to methionine utilizing 5-methyl-tetrahydrofolate as carbon donor. This inter-dependence is believed critical because having methyl-cobalamin in the reduced form ultimately dictates whether Folate can be utilized and stored in cells. Furthermore, with aging and/or defects in methionine synthase reductase (MTRR), it becomes more difficult to prevent increases in oxidized inactive methyl-cobalamin.

Adenosyl-cobalamin is the essential cofactor for the mitochondrial enzyme MCM that is required to convert methylmalonylCoA to succinylCoA. This conversion is essential for formation of the myelin sheath in neurons and to fuel the Krebs cycle to generate ATP since with aging, the ability to digest foods rich in B-12 and absorb/metabolize B-12 is impaired.

In present examples, the dosage of Vitamin B12 in the nutritional supplement composition is about 20 μg/day.

The term “Vitamin D” includes all functionally equivalent forms thereof.

Vitamin D plays an important role in prevention of osteoporosis it is also associated with a multitude of health disorders, when deficient, such as immune dysfunction, non-insulin dependent diabetes, periodontal disease, cardiovascular disease, proliferative skin diseases, muscular weakness and cancer.

Vitamin D regulates homeostasis and bone metabolism. However, it is also associated with protective effects against cancer. The anti-cancer effects of Vitamin D include prevention of DNA damage by promoting DNA repair and proper maintenance of telomere length control.

Vitamin D is primarily produced by a photochemical reaction in skin, using the energy of ultraviolet B radiation. Ultraviolet radiation in sunlight causes several types of DNA damage, immunosuppression and photoaging. The Vitamin D system in skin, together with metabolites of over-irradiation products, and Vitamin D receptors induce an adaptive response to UV that reduces DNA damage, inflammation and photocarcinogenesis. Vitamin D can also reduce thymine dimers, the major photoproduct found in human skin after UV exposure, and suppresses the accumulation of nitric oxide derivatives that lead to toxic reactive nitrogen species (RNS).

Vitamin D promotes expression of the DNA repair genes RAD50 and ATM, both of which are critical for mediating the signaling responses to DNA damage, especially DNA strand breaks (DSBs). Vitamin D also exerts these effects by inhibiting Cathepsin-L (CTSL) degradation of 53BP1 that is essential to first sense DSB-induced heterochromatin structural changes and second+ to activate ATM signaling required to recruit DNA DSB repair enzymes. Further, Vitamin D has chemopreventive and anti-aging properties.

Still further, Vitamin D induces transcription of the uridine phosphorylase gene (UPP1) that reduces concentration of dUTP and prevents DNA damage. Excessive dUTP can increase incorporation of uridine into DNA that is highly mutagenic.

It has been found that there is a positive association between Vitamin D status and increased telomere length. Without wishing to be bound by a single theory, it is believed to be related to its role in up-regulating repair of DSBs, which prevents loss of telomeres caused by terminal deletions of chromosomes. It is also believed that the role of Vitamin D in preventing DNA damage by promoting efficiency of DNA strand break repair is the most likely explanation for its cancer initiation prevention effects. It is further believed that the capacity of Vitamin D (calcitriol) of inducing cell differentiation and better control of cell replication is a plausible mechanism by which cancer growth and metastasis is inhibited.

In present examples, Vitamin D is in the form cholecalciferol (Vitamin D3). Cholecalciferol is converted by 25-hydroxylation in the liver to form 25[OH]D3, which subsequently is 1-hydroxylated by renal 1-α hydroxylase (CYP27b1) to form 1.25[OH]₂ D3 (Calcitriol). Calcitriol is the active form of Vitamin D shown to exert most of the health promoting activities in non-skin tissues including DNA strand break repair.

In present examples, the dosage of Vitamin D in the nutritional supplement composition can be about 2000 IU/day. It has been found that such a dose, or a lower dose of 600 IU/day, can increase telomerase activity or reduce oxidative damage to DNA in colorectal tissue.

The term “Zinc” includes all functionally equivalent forms thereof.

More than 300 enzymes and 1000 transcription factors are known to require Zinc for their activities. A significant portion of cellular Zinc is found in the nucleus where it is critically involved in maintaining genetic stability and in the process of gene expression via transcription factors that contain specific Zinc-finger regions that bind to DNA and control cell proliferation, differentiation and cell death. Zinc also plays a critical role in DNA damage prevention, DNA repair and DNA replication. Zinc is an essential cofactor or structural component for important antioxidant defense proteins and DNA repair enzymes, such as Cu/Zn SOD and OGG1, respectively, and can also affect activities of enzymes such as Human Betaine-Homocysteine Methyltransferase (BHMT) and 5-methyltetrahydrofolate-homocysteine methyltransferas (MTR) involved in methylation reactions in the Folate-methionine cycle. Zinc also induces the synthesis of metallothionein, which is a protein effective in reducing BHMT and MTR hydroxyl radicals and sequestering reactive oxygen species (ROS) produced in stressful situations, such as in type 2 diabetes, obesity and cancer. Dietary deficiencies in Zinc can contribute to single-strand and double-strand DNA breaks and oxidative modifications to DNA that increase risk for cancer development.

Maternal Zinc deficiency produces effects ranging from infertility and embryo/fetal death, to intrauterine growth retardation and teratogenesis. Major clinical problems resulting from Zinc deficiency include growth retardation, cell-mediated immune dysfunction and cognitive impairment. Zinc has been successfully used as a therapeutic modality for the management of acute diarrhea in children, Wilson's disease, the common cold and for the prevention of blindness in patients with age-related dry type of macular degeneration, and is very effective in decreasing the incidence of infection in the elderly. Zinc not only modulates cell-mediated immunity but is also an antioxidant and anti-inflammatory agent.

In present examples, Zinc is in the form of Zinc gluconate. Zinc gluconate is one of the most bioavailable and palatable forms of Zinc. In the examples, the dosage of Zinc in the formulation is about 20 mg/day. A dosage of 20 mg/day has been found to significantly reduce DNA damage biomarkers including micronuclei, DSBs and oxidized guanine. Dosages from 6 mg to 20 mg are particularly effective.

The term “Sulforaphane” includes all functionally equivalent forms thereof.

It has been found that Sulforaphane is a chemopreventive phytonutrient that can inhibit metabolic activation of carcinogens by phase I enzymes, enhance phase II enzymes involved in detoxification of carcinogens trapping of electrophiles and free radicals and protect of nucleophilic sites in DNA.

For example, Sulforaphane inhibits the phase I enzyme cytochrome P450 isoenzyme 2E1 (CYP2E1), which is responsible for activation of several carcinogens, including attenuating the genotoxicity of the carcinogen N-nitrosodimethylamine and exerting cardioprotective action by decreasing intracellular ROS production, increasing cell viability, and decreasing DNA fragmentation via the induction of antioxidants and phase II enzymes involved in the detoxification of xenobiotics.

Sulforaphane can facilitate protecting liver health not only from hepatotoxic chemicals but also from lifestyle-related factors such as high-energy food consumption. Also, Sulforaphane can cause significant HDAC and DNMT inhibition, leading to increased acetylation of histones and demethylation of tumor suppressor gene promoters that results in restoration of tumor suppressor gene expression and reduction of cells with DNA damage. Further, Sulforaphane enhances progerin clearance by stimulating proteasome and autophagy activity and reverses the phenotypic changes (e.g. DSBs, genomic instability, and nuclear shape abnormalities) that are the hallmarks of accelerated aging in Hutchinson-Gilford Progeria Syndrome cells. Progerin accumulation in the nuclear membrane and in the nucleus distorts the nuclear architecture and negatively effects nuclear processes including DNA replication and repair, leading to accelerated cellular aging and premature senescence.

Still further, Sulforaphane can protect normal cells from DNA damage and, at higher doses it can sensitize cancer cells to cytotoxic drugs. Sulforaphane can protect against the DNA damaging effects of (i) ionizing radiation and bleomycin by preventing induction of chromosome aberrations measured using the cytokinesis-block micronucleus (CBMN) assay, and (ii) five different mutagens namely, ethyl methanesulfonate, vincristine, colchicine, hydrogen peroxide and mitomycin C also measured also by CBMN assay. Further, Sulforaphane can promote elimination of DNA-damaged cells by apoptosis and protect against genotoxicity of aflatoxin-B1 measured as DNA adducts. Still further, Sulforaphane can damage DNA in cancer cells leaving normal cells intact.

In present examples, Sulforaphane is in the form of Broccoli sprout or seed extract (BSE) containing glucoraphanin (GR), a precursor of Sulforaphane (SF) and/or Broccoli sprout or seed extract (BSE) containing Sulforaphane (SF). In the examples, the dosage of Sulforaphane in the formulation is about 30 mg GR per day which corresponds to about 24 mg of SF per day. In other examples when present, the effective dosage is as low as 7 mg.

The term “Grape Seed Proanthocyanidins” includes all functionally equivalent forms thereof.

It has been found that Grape Seed Proanthocyanidin extract increases levels of alpha-tocopherol in red blood cell membranes. It has also been found that Grape Seed Proanthocyanidin can protect against UVB photocarcinogenesis of skin.

Grape Seed Proanthocyanidin facilitates maintaining nocturnal melatonin levels during the daytime. Advantageously, increased melatonin can offer both direct and indirect protection against a wide variety of DNA damaging agents by: (i) inhibition of metal-induced DNA damage; (ii) protection against non-radical triggers of oxidative DNA damage; (iii) activation of antioxidative enzymes; (iv) inhibition of pro-oxidative enzymes; and (v) boosting of the DNA repair machinery.

Grape Seed Proanthocyanidins have genome protective effects, such as enhancing the regenerative capacity of tissues and reducing the odds of carcinogenesis and/or accelerated aging. Also, Grape Seed Proanthocyanidins induce release of endothelial growth factor and have anti-microbial properties that can facilitate the closure and cure of skin wounds. Further, Grape Seed Proanthocyanidins can lower blood pressure in individuals with pre-hypertension and improve fasting insulin and insulin sensitivity.

In present examples, Grape Seed Proanthocyanidin is in the form of GSE. In the examples, the dosage of Grape Seed Proanthocyanidin in the formulation is about 280 mg/day. It has been found that dosages can be as high as 3000 mg/day. However, a dosage range of 80-300 mg/day has been found to be particularly effective in improving cardiovascular and antioxidant capacity parameters, including prevention of DNA oxidation.

Nutritional supplement compositions according to the present disclosure can be manufactured into a variety of forms, including solid and liquid dosage forms. Nonlimiting examples of such forms include tablets, caplets, liquid-filled soft capsules, suppositories, solutions and syrups.

In present examples, the nutritional supplement compositions are suitable for oral use, including, as tablets, lozenges, hard or soft capsules, aqueous or oily suspensions, emulsions, dispersible powders or granules, syrups or elixirs.

Oral compositions can have one or more coloring, sweetening, flavoring and/or preservative agents.

For a tablet formulation, suitable pharmaceutically acceptable excipients can include inert diluents such as lactose, sodium carbonate, calcium phosphate or calcium carbonate. A tablet formulation can also include: granulating and disintegrating agents such as corn starch or algenic acid; binding agents such as starch; lubricating agents such as magnesium stearate, stearic acid or talc; preservative agents such as ethyl or propyl p-hydroxybenzoate; or antioxidants such as ascorbic acid.

Tablet formulations can be uncoated or coated. A coating can modify disintegration of the tablet and the subsequent absorption of the active ingredient in the gastrointestinal tract, or to improve their stability and/or appearance. The tablet formulations can use conventional coating agents and procedures.

Compositions in the form of hard gelatin capsules can have the active ingredient mixed with an inert solid diluent, such as calcium carbonate, calcium phosphate, rice fiber, rice extract or kaolin.

Compositions in the form of soft gelatin capsules can have the active ingredient mixed with water or an oil, such as peanut oil, liquid paraffin or olive oil.

Aqueous suspensions can include, in some embodiments, the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia.

Aqueous suspensions can include, in other embodiments, the active ingredient in finely powdered form together with one or more dispersing or wetting agents. Dispersing or wetting agents include lecithin, condensation products of an alkylene oxide with a fatty acids such as polyoxethylene stearate, condensation product of ethylene oxide with long chain aliphatic alcohol like heptadecaethyleneoxycetanol, condensation product of ethylene oxide with partial esters derived from fatty acid and a hexitol such as polyoxyethylene sorbitol monooleate, and condensation product of ethylene oxide with partial ester derived from fatty acid and hexitol anhydride such as polyethylene sorbitan monooleate.

Aqueous suspensions can also include one or more: preservatives such as ethyl or propyl p-hydroxybenzoate, antioxidants such as ascorbic acid, coloring agents, flavoring agent, and/or sweetening agents such as sucrose, saccharine or aspartame.

The composition of the present disclosure can also be a solid edible product, a powdered edible product, a liquid edible product, a flowable edible product, a spoonable edible, a pourable edible product and a spreadable edible product or some mixture thereof.

In some embodiments, a powder can be mixed with a liquid, such as water or milk, to produce a liquid or slurry product. In other embodiments, a powder can be directly added to a food product.

The composition of the present disclosure can also be a beverage product. Beverage products include dairy based products and soy based products.

The edible compositions can be dairy based products, soy based products including drinks or beverages including a ready-to-drink liquid, a liquid produced from a soluble powdered product, breads and cereal based products like pasta or noodle based products and cereal bars, cakes, biscuits, ice creams, desserts, soups, porridge-type products, bar products, confectionery, snack foods, ready-to-eat meal products, pre-packed meal products, and dried meal products, soluble powdered products, and the like.

In other examples, the nutritional supplement compositions are suitable for topical use, including as creams, ointments, gels, or aqueous or oily solutions or suspensions.

In still other examples, the nutritional supplement compositions are suitable for administration by inhalation or insufflation, including as a finely divided powder or a liquid aerosol.

In yet other examples, the nutritional supplement compositions are suitable for parenteral administration, including as a sterile aqueous or oily solution for intravenous, subcutaneous, or intramuscular dosing or as a suppository for rectal dosing.

The nutritional supplement of the present disclosure is substantially free of Vitamin A and Vitamin B-1.

### EXPERIMENTAL

Customized culture media were prepared. The basal level for each selected nutrient was adjusted to reflect either (a) a background of deficiency in these nutrients that was predicted to increased DNA damage, or (b) a background Replete for these nutrients that was adequate for improved maintenance of DNA integrity. These Deplete and Replete media, respectively, were designed to mimic the impact of administering composition F1 (including Folate, Vitamin B12, Vitamin D, Zinc, Sulforaphane, and Grape Seed Proanthocyanidins) and/or another composition F2 (including Vitamin C, Vitamin E, selenium, carnosine, curcumin, hesperidin, lycopene, and lutein)—to a nutrient-Deplete, or Replete individual, on their genome integrity.

The following study design was applied for in vitro studies:

| Basal medium | Level and type of Supplementation | | | |
|---|---|---|---|---|
| DEPLETE | 0% | 30% F1 | 30% F2 | 30% F1 + F2 |
| DEPLETE | 0% | 100% F1 | 100% F2 | 100% F1 + F2 |
| REPLETE | 0% | 30% F1 | 30% F2 | 30% F1 + F2 |
| REPLETE | 0% | 100% F1 | 100% F2 | 100% F1 + F2 |

| | Media Condition Numbering |
|---|---|
| 1 | D 0% |
| 2 | D 30% F1 |
| 3 | D 30% F2 |
| 4 | D 30% F1 + F2 |
| 5 | D 0% (same as #1) |
| 6 | D 100% F1 |
| 7 | D 100% F2 |
| 8 | D 100% F1 + F2 |
| 9 | R 0% |
| 10 | R 30% F1 |
| 11 | R 30% F2 |
| 12 | R 30% F1 + F2 |
| 13 | R 0% (same as #9) |
| 14 | R 100% F1 |
| 15 | R 100% F2 |
| 16 | R 100% F1 + F2 |

D = Deplete nutrient background;
R = Replete nutrient background
F1 = formula1,
F2 = formula2,
F1 + F2 = formula1 + formula2.
0% = no supplement,
30% = 30% of optimal supplement dose,
100% = 100% of optimal supplement dose

The in vitro model involved 11-day cultures of human peripheral blood lymphocytes grown under conditions equivalent to clinical deficiency and clinical sufficiency in the micronutrients provided F1 and/or F1.

White blood cells (lymphocytes) isolated from whole blood of six healthy adult donors were maintained in Deplete or Replete culture medium with different conditions of F1 and/or F2 supplementation (at 0%, 30% or 100% of optimal dose). The genome integrity status of cells exposed to the different combinations of nutrients was then determined by comparing the frequencies of DNA damage biomarkers (micronuclei, nucleoplasmic bridges, nuclear buds) and telomere length in each culture condition, together with indicators of nuclear/cellular division, necrosis and apoptosis.

Eleven outcome measures were reported and analyzed including four biomarkers of DNA damage: Binucleates (BN) with micronuclei (MN), Total MN in BN, BN with Nucleoplasmic bridges (NPB), and BN with nuclear buds (NBud); four biomarkers of cell division: Mononucleates (Monos), Binucleates (BN), Multinucleates (Multis), and Nuclear division index (NDI, a value calculated based on frequency of Monos, BN and Multis); two markers of cell death: Apoptosis, and necrosis; and Telomere length.

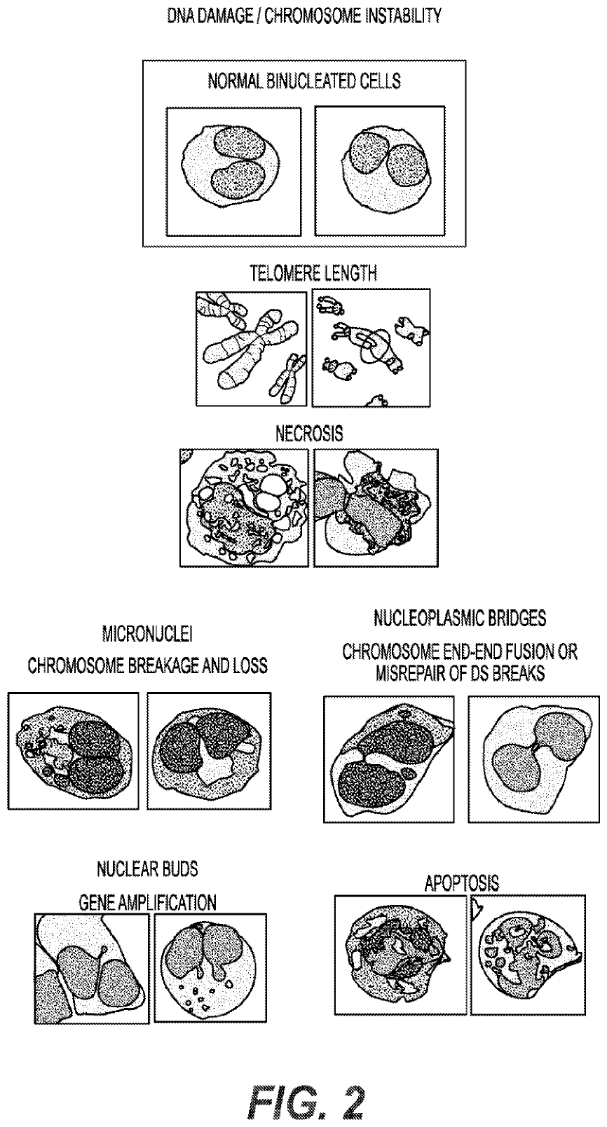

Examples of cellular damage at the DNA level that can occur without supplementation are shown in FIG. 2.

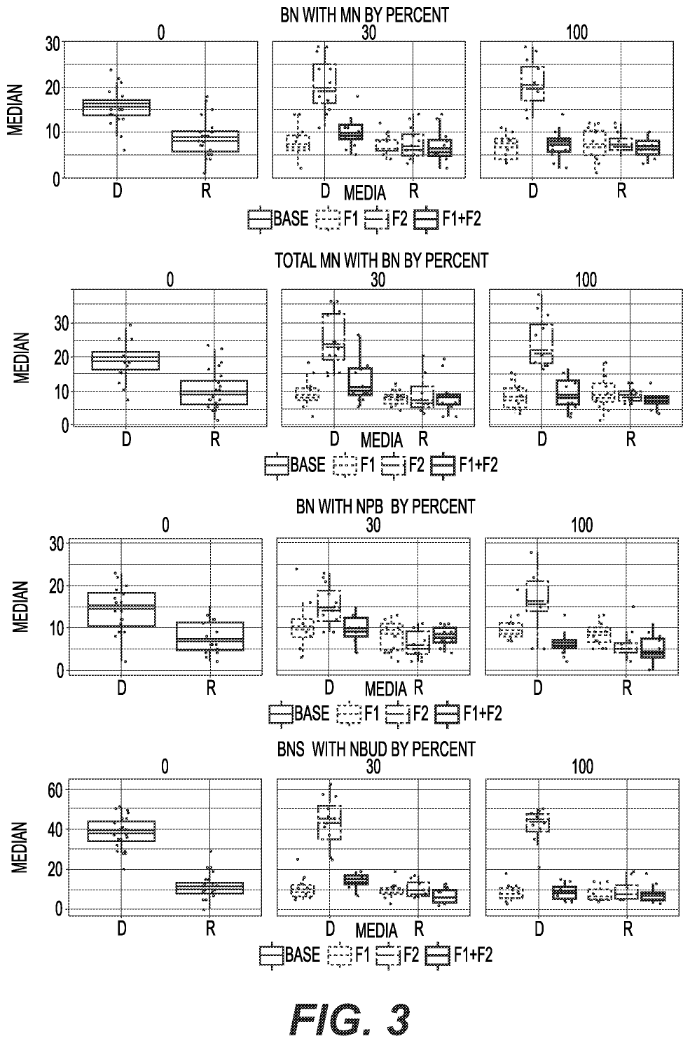

FIG. 3 summarizes the effect of supplementation with the F1, F2 and a combination of F1+F2 on BN with MN, BN with NPB and BN with NBuds at 0%, 30% or 100% of optimal dosage, in either Deplete (D) or Replete (R) basal medium. Statistical analyses were performed through Linear Mixed Effects Modeling.

Micronuclei (MN), Nucleoplasmic Bridges (NPB) and nuclear buds (NBud) are robust, and thus validated measures of DNA damage. Higher frequencies of each has been associated (individually and in combination) with nutritional deficiency and increased morbidities including cardiovascular disease, neurodegenerative conditions, and numerous cancers.

MN are indicative of chromosome breakage or loss. During mitosis, broken DNA fragments or whole chromosomes that fail to segregate properly are packaged by the cell into discrete nuclear membranes, separate to the main nuclei. Thus, these can be visualized and quantified as DNA damage events.

NPB originate from aberrant dicentric chromosomes formed by the misrepair of two uncapped or broken chromosomes, which can occur following loss of telomeric ends. NPB will break during mitosis, however in the CBMN-cyt assay cells NPB is blocked at the binucleated stage, allowing visualization (and quantification) of NPB as damage events.

NBud are formed when NPB are broken unevenly during mitosis, resulting in abnormal amplification of genetic material which the cell then expels from the nucleus into the cytoplasm via the process of nuclear budding. NBud are distinguished from MN because of their residual attachment to the nucleus.

“BN with MN” indicates the percentage of binucleated cells that contain one or more micronucleus. “Total MN” represents the actual number of MN scored, in total, in binucleated cells, in each respective data grouping.

Both measures showed greater frequency of MN in cells cultured in the un-supplemented Deplete media base compared to the Replete medium.

Supplementation had no effect on MN frequency at either 30% or 100% dosage in the Replete base (p>0.05). Supplementation reduced MN frequency to optimal levels with F1 (β=1.02, p<0.0001) or with F1+F2 (β=0.657, p=0.0043) in the Deplete medium, to a level similar to what can be seen in cells grown in Replete medium. Supplementation with F2 alone (β=−0.773, p=0.0008) significantly increased the DNA damage biomarker, over and above the “base” level seen in the Deplete media. Comparison of the median biomarker levels can be seen in FIG. 3.

“BN with NPB” indicates the percentage of binucleated cells that contain one or more NPB, in each respective data grouping.

Higher frequencies of NPB were recorded in the un-supplemented Deplete medium.

Supplementation had no effect at either 30% or 100% dosage in the Replete medium (p>0.05).

However, in the Deplete medium, supplementation with F1 (β=0.741, p=0.0059) or with F1+F2 (β=0.741, p=0.0059) reduced NPB frequency to optimal levels. Supplementation with F2 did not reduce NPB in cells grown in Deplete medium.

“BN with NBud” indicates the percentage of binucleated cells that contain one or more NBud, in each respective data grouping.

Considerably higher frequencies of NBud were recorded in the un-supplemented Deplete media base. Supplementation had no effect at either 30% or 100% dosage in the Replete base medium (p>0.05). However, in the Deplete base supplementation with F1 (β=1.26, p<0.0001) or with F1+F2 (β=0.844, p<0.0001) reduced NBud frequency to optimal levels. Supplementation with F2 did not reduce NBud in the Deplete base medium.

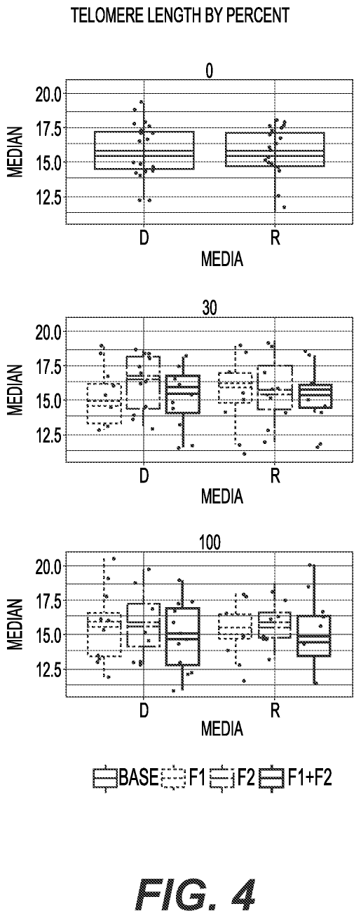

FIG. 4 shows the effect of supplementation with F1, F2 and a combination of F1+F2 at 0%, 30% or 100% of optimal dosage, in either Deplete or Replete basal medium.

Telomere length (TL) was measured by flow cytometry in viable cells at G0/G1 of the cell cycle. The result is the mean telomere content of cells from each condition, expressed as a value relative to a control cell line.

No difference in TL was observed between the un-supplemented Deplete and Replete conditions. Supplementation had no effect at either 30% or 100% dosage in the Deplete or Replete base media.

With respect to cytostasis, necrosis, apoptosis and NDI, FIG. 4 shows the effect of supplementation with F1, F2 and a combination of F1+F2 at 0%, 30% or 100% of optimal dosage, in either Deplete (D) or Replete (R) basal medium.

Cytostasis reflects the rate of cell division in each culture condition. This is quantified using the NDI, a value calculated from the relative frequencies of mononucleated cells (those that have not divided during the defined culture period), binucleated cells (once divided cells containing two nuclei), and Multinucleated cells (cells with 3 or more nuclei, indicating 2 or more rounds of cell division have occurred).

The NDI (Nuclear Division Index) is calculated using the following equation:

NDI=(M1+2M2+4M4)/N

where M1=number of mononucleated cells, M2=number of binucleated cells and M4=number of cells with more than 2 nuclei, and N is the total number of viable cells scored (excluding necrotic and apoptotic cells).

Mononucleated, Binucleated and Multinucleated cells.

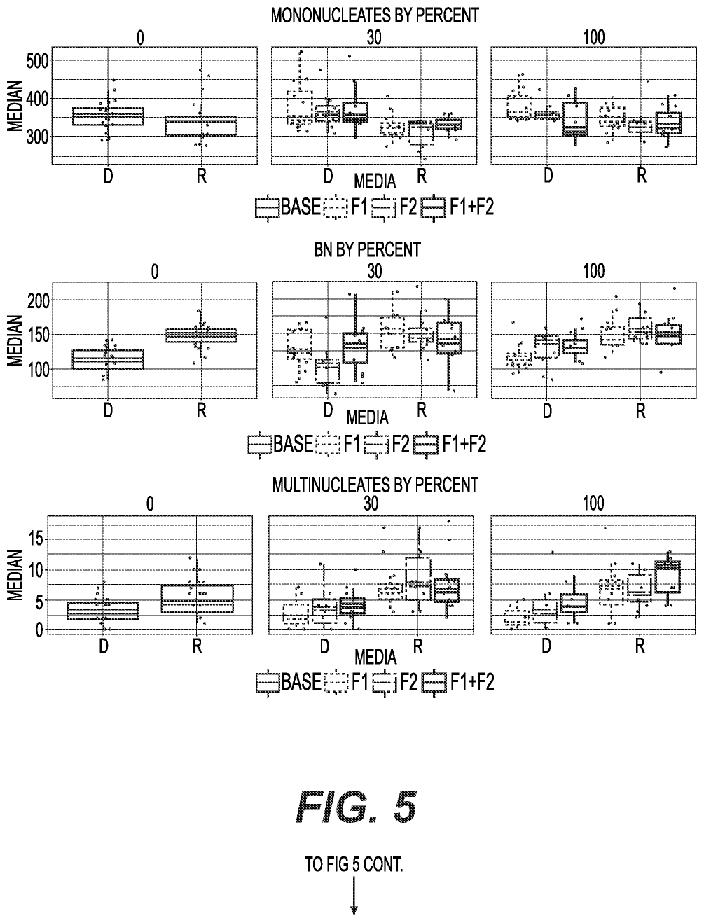

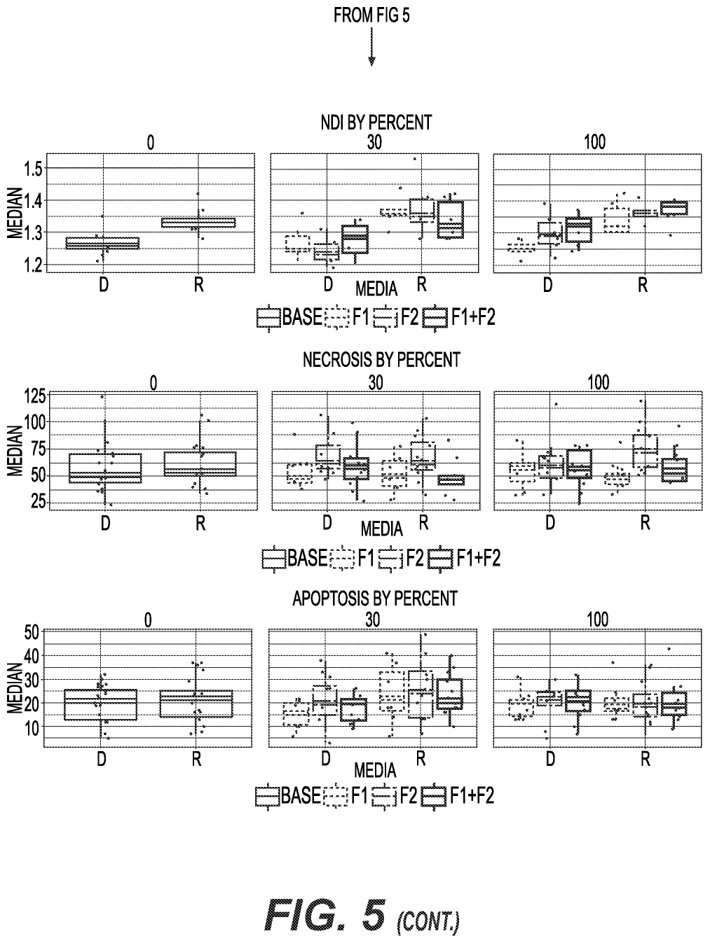

The left panels of the “Box and whisker” plots of FIG. 5 show the comparison between the 8 cultures in Deplete basal media, and the 8 cultures in Replete basal media. The observations for Monos, BN and Multis are consistent with expectation that cell division would be slower in the nutrient deficient cultures, and more prevalent in the nutrient Replete conditions.

For Monos, a main effect of F1 treatment was observed in the Deplete base condition (β=0.0697, p=0.0264). The interaction between media base and treatment was not significant, and as such little differences were expected when stratifying into either Deplete or Replete subgroups. For BN, a significant interaction was found between media base and F1+F2 treatment (p=0.027), thus it was expected that a different relationship would be assessed in either the Deplete or Replete media bases.

It was found that the combination of F1+F2 treatments was significantly different as compared with no formulation on frequency of Multinucleated cells (β=0.436, p=0.0045).

Necrosis is a form of cell death caused by damage to cellular membranes, organelles and/or critical metabolic pathways required for cell survival. Apoptosis, on the other hand, is when a cell undergoes programmed cell death. The latter is less likely to lead to an immune or inflammatory response.

It was found that basal media (D or R) and supplementation had minimal impact on necrosis. A slight increase was observed with F2 supplementation as compared with the base in the complete dataset (Main effect β=0.149, p=0.016). Stratification for media base showed significance for the F2 supplementation over the base in the Replete media only (β=0.290, p=0.00038).

It was also found that basal media (D or R) and supplementation had minimal impact on apoptosis. A slight increase was observed with F1, F2 and F1+F2 supplementation in the 30% dosage, however this was not significant.

A summary of the bioefficacy, plausibility and criticality is provided in the table below.

Summary Table of Bioefficacy, Plausibility and Criticality of Micronutrients in Formula #1

| Micronutrient | Bioefficacy | Plausibility | Criticality |
|---|---|---|---|
| Folic acid | High | high | High |
| Vitamin B12 | Medium | high | High |
| Vitamin D | High | medium | High |
| Zinc | High | high | High |
| Grape Seed Pro- | Medium | medium | Medium |
| anthocyanidins | | | |
| Sulforaphane | Medium | medium | Medium |

The studies conducted and the data suggest that a composition according to the present disclosure can improve DNA integrity in those who: (i) are deficient in one or more of the ingredients in the supplement due to malnutrition; (ii) are deficient in one or more of the ingredients in the supplement due to genetic defects in the absorption and metabolism of these micronutrients; (iii) are exposed to environmental genotoxins whose DNA damaging effects can be attenuated by the micronutrients in the supplements; and/or (iv) have genetic defects in DNA replication and repair that can be attenuated by the micronutrients in the supplement.

It will be apparent to those skilled in the art that various modifications and variations can be made in the present disclosure without departing from the spirit or scope of the present disclosure. Thus, it is intended that the present disclosure covers the modifications and variations of this disclosure provided they come within the spirit or scope of the appended claims and their equivalents.

What is claimed is:

1. A composition for oral delivery to a subject comprising a combination of: Folate, Vitamin B12, Vitamin D and Zinc.

2. The composition according to claim 1, further comprising Grape Seed Proanthocyanidins and Sulforaphane.

3. A method to increase DNA replication comprising administering the composition of claim 2.

4. A method to increase cellular efficiency of DNA repair by comprising administering the composition of claim 2.

5. A method to induce a cell to increase cellular methylation of DNA comprising administering the composition of claim 2.

6. A method to increase telomere maintenance comprising administering the composition of claim 2.

7. A method to reduce cellular uridine DNA mutations comprising administering the composition of claim 2.

8. A method to reduce abnormal methylation of DNA in a cell, the method comprising administering the composition of claim 2.

9. A method to induce a cellular reduction in DNA strand breaks and base damage comprising administering the composition of claim 2.

10. A method for reducing inflammation by preventing micronucleus formation comprising administering the composition of claim 2.
